(19) United States
(12) Patent Application Publication
(10) Pub. No.: US 2008/0233612 A1
(43) Pub. Date: Sep. 25, 2008

(54) Fungal Hosts for Expression of Recombinant Products

(76) Inventors: Helena Nevalainen, New South Wales (AU); Valentino Setoa Te'o, New South Wales (AU); Peter Leonard Bergquist, Auckland (NZ); Roberta Farrell, Hamilton (NZ)

Correspondence Address: BARNES & THORNBURG LLP, P.O. BOX 2786, CHICAGO, IL, 60690-2786, US

(21) Appl. No.: 10/573,343

(22) PCT Filed: Sep. 23, 2004
(86) PCT No.: PCT/AU04/01303

§ 371 Date: Jul. 13, 2007

(30) Foreign Application Priority Data
Sep. 24, 2003 (AU) 2003905228

(51) Int. Cl. C12P 21/00; C12N 1/14; C12N 1/15

(52) U.S. Cl. 435/69.1; 435/254.1; 435/254.11

(57) ABSTRACT

An isolated mutant Ophiostoma species having enhanced protein excretion capability as compared with its parent strain cultured under similar conditions.

### TECHNICAL FIELD

The present invention relates to fungal hosts having enhanced protein excretion.

### BACKGROUND ART

The ascomycete Ophiostoma spp. comprise common sap-staining fungi such as O piceae that cause discoloration of wood. Some albino variants of O. flocossum have been used as biological control agents to prevent sap-staining (Harrington et al., 2001. Mycologia, 93:110-135). A fungus capable of invading wood via the parenchyma cells must secrete enzymes outside the growing mycelium in order to utilize the non-structural components of sapwood (eg. sugars, proteins and extractives). Secreted enzymes such as lipases (Brush et al., Bio. Med. Chem. 7:2131-2138, 1999) and proteinases (Abraham and Breuil, Enzyme Microb. Technol. 18:133-140, 1996) from Ophiostoma have been studied in some detail. However, in addition to these studies, very little has been published about biochemical and genetic properties of Ophiostoma.

The present inventors have developed Ophiostoma mutants as hosts for expression of industrially important enzymes and biomolecules. Ophiostoma mutants can be used for new expression systems.

### DISCLOSURE OF INVENTION

In a first aspect, the present invention provides an isolated mutant Ophiostoma species having enhanced protein excretion capability as compared with its parent strain cultured under similar conditions.

Preferably, the isolated Ophiostoma species is Ophiostoma floccosum. More preferably, the Ophiostoma floccosum is selected from mutant strains J2026MQ.1.1, J2026MQ.1.2, J2026MQ.2.1, J2026MQ.3.1, J2026MQ.4.1, J2026MQ.5.1 and J2026MQ.5.5, as herein defined.

Mutants J2026MQ.1.1, J2026MQ.1.2, J2026MQ.5.1 and J2026MQ.5.5 were deposited at the National Measurement Institute (NMI) (formally Australian Government Analytical Laboratory (AGAL)) on 17 Sep. 2004 under accession numbers as set out below.

| Mutant designation | NMI Accession Number |
|---|---|
| J2026MQ.1.1 | NM04/42878 |
| J2026MQ.1.2 | NM04/42879 |
| J2026MQ.5.1 | NM04/42880 |
| J2026MQ.5.5 | NM04/42881 |

In a more preferred embodiment, the isolated Ophiostoma species is Ophiostoma floccosum strain J2026MQ.1.1, or J2026MQ.1.2 or J2026MQ.5.1 or J2026MQ.5.5 or any of their descendants or mutant thereof.

The present inventors have found that mutant strains having enhanced protein excretion capability are particularly suitable for receiving and harbouring expression vectors and producing recombinant products.

Preferably, the protein is an enzyme. More preferably, the enzyme is selected from protease, amylase, lipase, glucoamylase, β-galactosidase, and β-glucosidase.

Preferably, the mutant Ophiostoma species according to the present invention is characterised by:

(a) one nucleus per conidium/blastospore;
(b) conidia having mean spore size no less than about 2-3 μm in diameter; and
(c) capable of secreting at least about two times more of a selected protein into culture medium when compared to the secretion of the parent strain grown under similar conditions.

Preferably, the mutant is capable of secreting at least about three times more of a selected protein when compared to the secretion of the parent strain grown under similar conditions.

Preferably, the selected protein is a proteinase.

The isolated Ophiostoma species has preferably undergone modification/selection to alter its ability to carry out or perform protein secretion.

It has been found by the present inventors that modifying/altering DNA by random mutagenesis creates a more adaptable Ophiostoma species to act as a host for gene expression.

Mutant Ophiostoma species according to the present invention differ from naturally occurring Ophiostoma species in their enhanced ability to excrete proteins into the cultivation medium.

In a second aspect, the present invention provides use of a modified fungal species according to the first aspect of the present invention in an industrial process such as pulping, bleaching, recombinant protein production, and any other suitable process utilizing microbial production or action.

An advantage of using a fungal species (Ophiostoma) instead of bacteria or yeasts is that Ophiostoma is naturally living on wood for in situ delivery of selected gene products.

The present invention is particularly suitable for developing modified fungal species which are capable of excreting extracellular components such as enzymes that can be applied to processes either as liquid form from submerged fermentation or delivered in situ by the fungus growing on a particular material in solid state.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of the claims of the invention.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the present invention may be more clearly understood, preferred forms will be described with reference to the following drawings and examples.

### BRIEF DESCRIPTION OF THE DRAWINGS

FIG. 1 shows SDS-PAGE separation of the proteins secreted by Ophiostoma J2026 and selected first round mutants. M, molecular weight markers. Lane 1, strain J2026; lanes 2-9, strains J2026MQ.1.1-J2026MQ.1.8, respectively.

FIG. 2 shows PCR analysis of Ophiostoma colonies following protoplast transformation. L, 1 kb plus DNA ladder, 1, control plasmid containing dsRed gene fragment, 2 and 3, represent non-transformant strains J2026MQ.5.1 and J2026MQ.5.5, respectively, lanes 4 to 18, represent transformants which survived the hygromycin B selection. Only transformants 4, 9, 10, 11 and 15 have the 0.7 kb dsRed gene fragment amplified.

### MODE(S) FOR CARRYING OUT THE INVENTION

In order to obtain the present invention high secreting Ophiostoma mutants were made and the second step was to develop an effective transformation system for Ophiostoma useful for one or more of three purposes: (i) introduction of novel properties in the fungus for biological control; (ii) introduction of novel enzyme activities such as ligninases in the fungus for biopulping purposes, and (iii) development of Ophiostoma as a novel system for the expression of a variety of industrially important gene products, either homologous or heterologous (enzymes, biopharmaceuticals, etc).

Basic requirements for a potential expression host strain, before commencing with biolistic transformation, are: (i) preferably one nucleus per blastospore/conidium; (ii) occurrence of (blasto)spores with a minimum length and diameter of 2-3 μm; and (iii) sensitivity to a potent antibiotic (eg. hygromycin B) at a manageable level (eg. 50 to 200 U/ml). Experiments on Ophiostoma floccosum strains J2026MQ.1.1, J2026MQ.1.2, J2026MQ.2.1, J2026MQ.3.1, J2026MQ.4.1, J2026MQ.5.1 and J2026MQ.5.5 display necessary features and therefore have been chosen as the preferred hosts for further development.

### Materials and Methods

In addition to promoter and terminator sequences, the expression cassette may contain, a signal sequence from a heterologous protein functional in Ophiostoma (for example T. reesei CBHI secretion signal sequence) or a signal sequence from the secreted host serine protease, lipase, amylase or glucoamylase enzyme, as well as a multiple cloning site for inserting any foreign DNA. Tests carried out to determine promoter strength as well as secretion signal sequence function involved the use of a gene encoding a fluorescent foreign protein such as dsRed1-E5 (Terskikh et al., Science, 290:1585-1588, 2000) fused to the different signal sequences and promoters. The best promoter-secretion signal sequence combinations were then used in future protein expression work using the high secreting O. floccosum host strain(s) described below.

### Screening for High Secretors

Secretion capacity of filamentous fungi can be improved by random mutagenesis and screening for enhanced and favourable characteristics. This procedure involves the treatment of fungal spores with physical (UV) or chemical mutagens, followed by screening for better secretors using a plate assay.

A 20 ml spore suspension of Ophiostoma floccosum (strain J2026) was placed on a 20 cm glass Petri dish at a concentration of 5×10⁸ spores/ml and irradiated at 254 nm UV-light for a period of 0 to 8 min. One ml samples were removed at one minute intervals, diluted in 0.9% NaCl, 0.01% Tween-80 solution and plated on Potato Dextrose Agar+0.1% Triton X100-plates for counting. Colonies from the 5-50% survival level were picked and patched onto minimal agar plates (Penttilä et al., Gene 61: 155-164, 1987) supplemented with soy hydrolysate (1.5% w/v) and insoluble starch (3% w/v) for the screening of starch-degrading enzymes (e.g. amylase, glucoamylase). The presence of clearing halos around the colonies indicates Starch degrading activity. Relative size of the hydrolysis halo around a mutant colony gives an indication of enzyme secretion when compared to the nonmutagenized parent. It will be appreciated that other screening means known to the art can also be used.

Colonies showing considerably larger halos when compared to the parent strain were grown in shake cultures in a medium promoting production of a wide variety of hydrolytic enzymes.

The basal liquid medium for general protein production contained 15 g KH₂PO₄, 5 g (NH₄)₂SO₄, 10 ml 100× trace elements (100 mg FeSO₄×7H₂O, 20 mg MnSO₄×H₂O, 20 mg ZnSO₄×7H₂O, 40 mg CoSO₄×7H₂O in 200 ml MQ water) and MQ water to a final volume of 1 litre. The pH was adjusted to 6.5. The basal medium was divided into 50 ml portions in 250 ml conical flasks and soybean flour (consisting of 52% protein, 1% fat) was added to each flask to a final concentration of 1.5% (w/v) for induction of e.g. lipase and protease production. The carbon source used for amylase induction was 3% (w/v) filter sterilized potato soluble starch (Sigma Chemicals). Each flask/was inoculated with 10⁸ spores and cultivated for 5 days at 28° C. and 250 rpm.

Enzymes secreted into the cultivation medium were assayed using previously published methods. Protease activity was monitored as in (Lovrien et al. J. Appl. Biochem. 7:258-272, 1985) and the method for amylase and glucoamylase activity has been described in Bailey and Nevalainen (Enzyme Microb Technol. 153-157, 1981). SDS-PAGE analysis of secreted proteins from the culture supernatants was carried out in 12.0% (w/v) and 4-20% (w/v) gradient acrylamide gels according to Laemmli (Nature 227, 680-685, 1970).

### Biolistic and Protoplast Transformations

Transformation was carried out using particle bombardment of fungal spores as outlined in Hazell et al. (Lett. Appl. Microbiol. 30:282-286, 2000) using a hepta-adaptor device that allows delivery of DNA into the conidia from seven barrels, thereby increasing the transformation efficiency (Te'o et al., J. Microbiol. Methods, 51:393-399, 2002). Seven portions of 7 to 14 days old spores (1×10⁷-1×10⁸ in 0.9% NaCl, 0.01% Tween 80 solution) were plated appropriately on PDA plates for hygromycin selection or MM plates containing acetamide as the sole nitrogen source for the selection of transformants (Penttliä et al., 1987) to align with the seven barrels of the Hepta Adaptor, and left to dry. Precipitation of circular and linear DNA onto tungsten particles (0.7 μm mean diameter, Bio-Rad) was carried out as described in Hazell et al., (2000) with some minor modifications. For example, 100 μl of tungsten beads were used instead of 50 μl per DNA sample and accordingly, the amount of 2.5 M CaCl₂ and 0.1 M Spermidine were increased to 100 and 40 μl from 50 and 20 μl, respectively during the precipitation step. Following precipitation, the mixture of tungsten particles coated with DNA were resuspended in 80 μl of 100% Ethanol, of which 10 μl (×7) samples were removed and used for bombardment as described below. DNA concentrations of 1000 ng (×7) were used in the experiments.

For bombardment, 10 μl of DNA-coated tungsten particles were loaded onto seven macrocarrier disks sitting in the seven slots of the Hepta Adaptor holder and left to dry. A single rupture disk of 1350 psi strength was placed on the top to temporarily block the helium gas from entering the seven barrels of the Hepta Adaptor instrument. When dried, the Hepta Adaptor holder containing DNA-coated tungsten beads was placed inside the PDS-1000/He system chamber directly below the Hepta Adaptor device. PDA plates containing conidia for bombardment were placed at a target distance of 3 cm.

Following bombardment, PDA plates were incubated at 28° C. for 4-6 hr before overlaying them with 10 ml of PDA containing hygromycin B to a final concentration of 60 U/ml or 90 μg/ml of G418.

Transformations using protoplasts were carried out as described in Wang et al. Mycol. Res. 103:77-80, 1999, with minor modifications. Briefly, a 100 ul protoplast solution (total of 1×10⁸) was gently mixed with 5-20 μg of transforming DNA and left on ice for 30 mins before plating out using a 7 mL RM (5.0% yeast extract, 2.0% glucose, 0.2% KH₂PO₄) agar overlay onto RM agar plates containing 60 U/mL of Hygromycin B. Plates were left at 28° C. until colonies emerged and were picked and screened for the presence of the transforming DNA using PCR.

### Results

### Identification and Improvement of Ophiostoma Strains Suitable as Expression Hosts by SDS-PAGE Analysis of Secreted Proteins

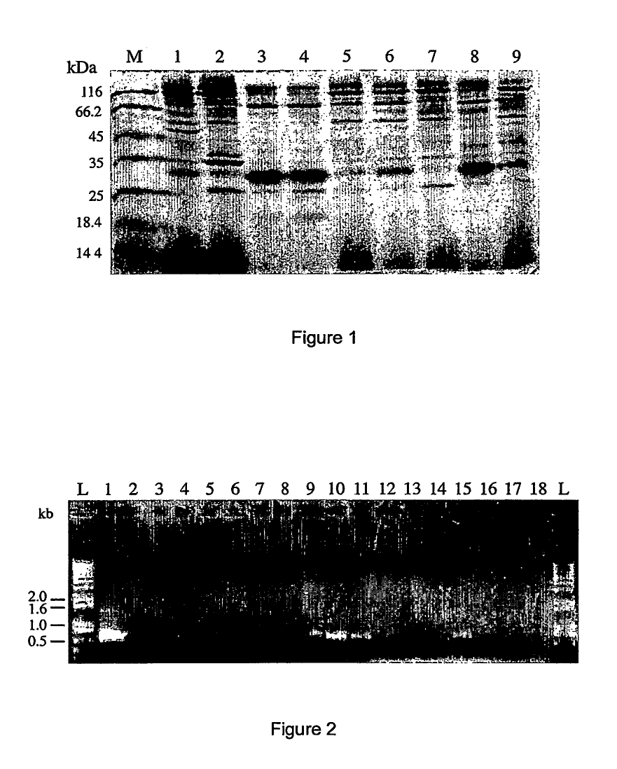

SDS-PAGE of secreted proteins was initially applied to recognize good protein secretors suitable for the development of expression hosts and to identify the most highly secreted proteins to isolate strong gene promoters driving their synthesis (discussed above). In order to profile the proteins secreted by Ophiostoma strains J2026 and J2026MQ.1.1-J2026MQ.1.8, culture supernatants were analysed on a 12% SDS-PAGE gel and stained with Coomassie stain (FIG. 1).

In order to develop the strains further, screening for strains with increased protein secretion and/or modified enzyme profile after UV mutagenesis was carried out. Plate assays were used for the screening of mutants, for example, with improved starch-degrading activity.

With strain J2026, nine mutant colonies with potentially increased starch degrading activity were picked from the screening plates and grown in liquid culture as described in Materials and Methods. Glucoamylase and protease activity as well as total secreted protein were assayed from the culture supernatants (Table 1).

TABLE 1
Glucoamylase (GA) and protease activity of first round UV mutants of
Ophiostoma J2026.

| | | Protease | Total protein |
|---|---|---|---|
| Strain | GA nkat/ml | Units* | (mg/ml) |
| J2026 (parent) | 10.3 | 4.43 | 0.228 |
| J2026MQ.1.1 | 36.83 | 0.25 | 0.268 |
| J2026MQ.1.2 | 18.4 | 13.25 | 0.236 |
| J2026MQ.1.3 | 18.23 | 13.03 | 0.213 |
| J2026MQ.1.4 | 13.35 | 4.97 | 0.220 |
| J202MQ.1.5 | 11.91 | 7.12 | 0.287 |
| J202MQ.1.6 | 10.48 | 0.48 | 0.200 |
| J2026MQ.1.7 | 9.99 | 13.41 | 0.205 |
| J2026MQ.1.8 | 9.53 | 5.81 | 0.246 |
| J2026MQ.1.9 | 7.41 | 2.86 | 0.269 |

*(as in Lovrien et al. 1985)

The mutant J2026MQ.1.1 showed about three fold increase in the glucoamylase activity and 18 fold decrease in the protease activity when compared to the nonmutagenised parent J2026. In addition, secretion of the total protein was increased by 14% in J2026MQ.1.1. In the mutant J2026MQ.1.5, the glucoamylase and protease activities were slightly increased when compared to the parent J2026, however, the total secreted protein was increased by 25%. These examples show that total protein secretion and protein profiles of Ophiostoma can be modified by random mutagenesis. Low protease producing strains are of special interest as expression host for foreign gene products.

FIG. 1 shows the proteins secreted by the mutants in Table 1, separated by SDS-PAGE highlighting the different protein profiles.

Protease Enzyme Activity Secretion Profiles from Ophiostoma

One important aspect of foreign protein production is proteolytic degradation of the heterologously produced protein by the host proteinases. Having first hand knowledge of the host's proteolytic profile (eg. secreted proteinases) can assist in the correct decision making in which expression host best suits the gene to be expressed. Selected O. floccosum mutant strains plus the parent were cultivated in minimal medium (Penttilä et al., 1987, Gene. 61:155-164) containing starch as a carbon source for 5 days at 28° C. Proteolytic enzyme activities determined from the culture supernatants are summarized in Table 2. According to the results in Table 2, mutant J2026MQ.1.2 secretes proteins which have strong metallo-like proteinase activity at pH 5, and high serine-like (Chymotrypsin) proteinase activity at pH 8. Culture supernatants were also analyzed on 1D PAGE gels and J2026MQ.1.2 was found to secrete high amounts of a ˜30 kDa protein when compared to the parent and to other mutant strains. This 30 kDa protein's N-terminal sequence was determined and found to show similarity to serine-like proteinases from other filamentous fungi. When compared to the parent as well as to J2026MQ.1.2, the mutants J2026MQ.1.1, J2026MQ.2.1, J2026MQ.3.1, J2026MQ.4.1 and J2026MQ.5.1 had higher glucoamylase activities which appeared to be coming from secreted proteins running at molecular weights of 70-80 kDa (also seen in FIG. 1). Importantly, the 30 kDa protein (a putative serine proteinase) mentioned above is not present in these high glucoamylase-producing mutants.

Table 2. Summary of secreted proteolytic enzyme activities from selected Ophiostoma floccosum strains. J2026 is the parent strain and J2026MQ.1.1, J2026MQ1.2, J2026MQ2.1, J2026MQ3.1, J2026MQ4.1 and J2026MQ5.1 were all mutant strains. Proteinase enzyme activities are represented according to ++++, strong; +++, high; ++, medium; +, low; −+, weak; and −−, no activity.

TABLE 2

| | Strain | | | | | | | | | | | | | |
|---|---|---|---|---|---|---|---|---|---|---|---|---|---|---|
| | J2026 | | J2026MQ.1.1 | | J2026MQ.1.2 | | J2026MQ.2.1 | | J2026MQ.3.1 | | J2026MQ.4.1 | | J2026MQ.5.1 | |
| Protease type | pH 5 | pH 8 | pH 5 | pH 8 | pH 5 | pH 8 | pH 5 | pH 8 | pH 5 | pH 8 | pH 5 | pH 8 | pH 5 | pH 8 |
| Metallo | +++ | +++ | +++ | ++ | ++++ | ++ | ++ | ++ | ++ | ++ | +++ | ++ | +++ | ++ |
| Serine | | | | | | | | | | | | | | |
| Chymotrypsin | −−/+ | ++ | −− | −− | + | +++ | −− | −− | −− | −− | −− | −− | −− | −− |
| Elastase | −− | −− | −− | −− | −− | −− | −− | −− | −− | −− | −− | −− | −− | −− |
| Subtilisin | −− | −− | −− | −− | −− | −− | −− | −− | −− | −− | −− | −− | −− | −− |
| pH | 5 | 7.6 | 5 | 7.6 | 5 | 7.6 | 5 | 7.6 | 5 | 7.6 | 5 | 7.6 | 5 | 7.6 |
| Trypsin | −− | −− | −− | −− | −− | −− | −− | −− | −− | −− | −− | −− | −− | −− |
| pH | 5 | 7.5 | 5 | 7.5 | 5 | 7.5 | 5 | 7.5 | 5 | 7.5 | 5 | 7.5 | 5 | 7.5 |
| Cysteine | −− | −− | −−/+ | + | −− | −− | ++ | +++ | ++ | +++ | ++ | +++ | ++ | +++ |

Proteinase enzyme activities are represented according to ++++, strong; +++, high; ++, medium; +, low; −+, weak; and −−, no activity.

### Transformation of Ophiostoma by Biolistic Bombardment

The average transformation efficiency of Ophiostoma was 4 transformants per microgram of DNA using 500 ng of DNA (hygB marker), 1350 psi and 3 cm target distance. Following bombardment, the transformants were tested on PDA containing 150 units per ml of hygromycin B and 90 μg per ml of G418 or identified by colony growth on minimal medium agar plates containing acetamide as the sole nitrogen source.

### Protoplast Transformation of Ophiostoma

Using the protoplast approach, approximately 1 in 7.3 colonies had the transforming DNA integrated into the Ophiostoma genome (FIG. 2). A total of 73 transformants which grew on hygromyin B containing plates were picked and genomic DNA from ten transformants showed to have the ˜0.7 kb dsRed gene fragment integrated into the genomic DNA (FIG. 2).

### SUMMARY

The present inventors have devised an Ophiostoma system for efficient expression of selected gene products. The system comprises promoter(s) functional in Ophiostoma, suitable transformation markers and transformation of a suitable Ophiostoma strain by biolistic bombardment and by protoplast transformation. The present inventors have shown that the amount of secreted protein of a fungal strain can be increased and protein profiles modified following UV-light mutagenesis. Mutagenesis also allows for the production of host strains with different background enzyme profiles. Enhanced secreting and low protease mutant strain(s) can be used as expression hosts in the production of industrially important molecules or proteins. The transformed mutant strain can be subjected to additional rounds of UV-light mutagenesis to further improve desired characteristics.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An isolated mutant Ophiostoma species having enhanced protein excretion capability as compared with its parent strain cultured under similar conditions.

2. The mutant according to claim 1 wherein the Ophiostoma species is Ophiostoma floccosum.

3. The mutant according to claim 2 selected from the group consisting of mutant strains J2026MQ.1.1, J2026MQ.1.2, J2026MQ.2.1, J2026MQ.3.1, J2026MQ.4.1, J2026MQ.5.1 and J2026MQ.5.5, as herein defined.

4. The mutant according to claim 2 selected from the group consisting of deposit accession numbers NM04/42878, NM04/42879, NM04/42880, NM04/42881, their progeny, and mutants thereof.

5. The mutant according to claim 1 capable of receiving and harbouring an expression vector and producing a recombinant product.

6. The mutant according to claim 1 wherein the protein is an enzyme.

7. The mutant according to claim 6 wherein the enzyme is selected from the group consisting of protease, amylase, lipase, glucoamylase, β-galactosidase and β-glucosidase.

8. A mutant Ophiostoma species is characterised by:
one nucleus per conidium/blastospore;
conidia having mean spore size no less than about 2-3 μm in diameter; and
capable of secreting at least about two times more of a selected protein into culture medium when compared to the secretion of the parent strain grown under similar conditions.

9. The mutant according to claim 8 capable of secreting at least about three times more of a selected protein when compared to the secretion of the parent strain grown under similar conditions.

10. The mutant according to claim 9 wherein the selected protein is a proteinase.

11. A method to provide microbial production or action in an industrial process selected from the group consisting of pulping bleaching and recombinant protein production the method comprising:
(a) providing an isolated mutant Ophiostoma species according to claim 1; and
(b) using the protein excreted by the mutant Ophiostoma species in the industrial process.

12. A method to provide microbial production or action in an industrial process selected from the group consisting of pulping, bleaching and recombinant protein production, the method comprising:
(a) providing a mutant Ophiostoma species according to claim 8; and
(b) using the selected protein excreted by the mutant Ophiostoma species in the industrial process.

13. The method of claim 11 wherein the protein is an enzyme.
